(19) United States
(12) Patent Application Publication
(10) Pub. No.: US 2022/0348880 A1
(43) Pub. Date: Nov. 3, 2022

(54) Xenogen-Free Mesenchymal Stem Cell Compositions and Methods of Use

(71) Applicant: THE TEXAS A&M UNIVERSITY SYSTEM, College Station, TX (US)

(72) Inventors: Ashlee Watts, College Station, TX (US); Aileen Rowland, College Station, TX (US)

(21) Appl. No.: 17/638,269

(22) PCT Filed: Aug. 30, 2019
(86) PCT No.: PCT/US19/49088

§ 371 Date: Feb. 25, 2022

(51) Int. Cl. C12N 5/0775; A61K 35/28; A61K 9/00

(52) U.S. Cl. CPC C12N 5/0663; A61K 35/28; A61K 9/0019

(57) ABSTRACT

The data presented herein relates to therapeutic compositions of mesenchymal stem cells (MSCs). In particular, pharmaceutically acceptable MSC compostions are xenogen-free and do not have immunological adverse effects. Mesenchymal stem cells expanded in a cell culture media comprising bone marrow supernatant produce xenogen-free mesenchymal stem cells. Such xenogen-free MSC compositions improve therapy for medical conditions including, but not limited to, osteoarthritis, cardiovascular disorders and/or diabetes.

### FIELD OF THE INVENTION

The present invention relates to therapeutic compositions of mesenchymal stem cells (MSCs). In particular, pharmaceutically acceptable MSC compositions are xenogen-free and do not have immunological adverse effects. Mesenchymal stem cells expanded in a cell culture media comprising bone marrow supernatant produce xenogen-free mesenchymal stem cells. Such xenogen-free MSC compositions improve therapy for medical conditions including, but not limited to, osteoarthritis, cardiovascular disorders, and/or diabetes.

### BACKGROUND

The most commonly used culture media supplement for mesenchymal stem cell (MSC) isolation and expansion is fetal bovine serum (FBS), which causes xenogeneic immune reactions in all species but bovine. While chemically derived FBS-free products are commercially available, none of these products result in an unaltered and optimal MSC product. This is because many of the biochemical factors present in FIBS are as of yet unknown and are therefore difficult to replace.

What is needed in the art is a substitute for FBS in MSC culture media that does not have adverse reactions and contains all the necessary biochemical factors to produce xenogeneic-free, viable MSCs.

### SUMMARY OF THE INVENTION

The present invention relates to therapeutic compositions of mesenchymal stein cells (MSCs). In particular, pharmaceutically acceptable MSC compositions are xenogen-free and do not have immunological adverse effects. Mesenchymal stem cells expanded in a cell culture media comprising bone marrow supernatant produce xenogen-free mesenchymal stem cells. Such xenogen-free MSC compositions improve therapy for medical conditions including, but not limited to, osteoarthritis, cardiovascular disorders and/or diabetes.

In one embodiment, the present invention contemplates a method, comprising: a) collecting a bone marrow sample from a subject; b) centrifuging said bone marrow sample to separate a first layer comprising mesenchymal stem cells and a second layer comprising a bone marrow supernatant; c) adding said bone marrow supernatant to a cell culture media, wherein said media is xenogen-free; d) proliferating said mesenchymal stem cells in said xenogen-free cell culture media, to create an expanded xenogen-free mesenchymal stem cell population. In one embodiment, the expanded xenogen-free mesenchymal stem cell population does not contain a fetal bovine serum immunogen. In one embodiment, the method further comprises incorporating said expanded xenogen free mesenchymal stem cell population into a pharmaceutically acceptable composition. In one embodiment, the subject is a mammal selected from the group consisting of human, equine, caprine, bovine and ovine.

In one embodiment, the present invention contemplates a method, comprising: a) providing; i) a subject comprising an osteoarthritis injury; and ii) a pharmaceutically acceptable composition comprising an expanded xenogen-free mesenchymal stem cell population; b) administering said pharmaceutically acceptable composition to said subject, wherein said osteoarthritis injury is at least partially healed. In one embodiment, the osteoarthritis injury completely heals within one year after said administering. In one embodiment, the administering comprises an injection selected from the group consisting of an intra-articular injection, an intramuscular injection an extracellular tendon injection and an intravenous injection. In one embodiment, the completely healed subject returns to pre-injury work or performance. In one embodiment, the subject is a mammal selected from the group consisting of human, equine, caprine, bovine and ovine. In one embodiment, the expanded xenogen-free mesenchymal stem cell population does not contain a fetal bovine serum immunogen. In one embodiment, the expanded xenogen-free mesenchymal stem cell population contains a bone marrow supernatant. In one embodiment, the osteoarthritis injury includes but is not limited to a hip joint injury, a knee joint injury, an ankle joint injury, an elbow joint injury, a finger joint injury, and a toe joint injury.

In one embodiment, the present invention contemplates a method, comprising: a) providing; i) a subject comprising a cardiovascular injury; and ii) a pharmaceutically acceptable composition comprising an expanded xenogen-free mesenchymal stem cell population; b) administering said pharmaceutically acceptable composition to said subject, wherein said cardiovascular injury is at least partially healed. In one embodiment, the cardiovascular injury completely heals within one year after said administering. In one embodiment, the administering comprises an injection selected from the group consisting of an intra-articular injection, an intramuscular injection, an extracellular tendon injection, an intravenous injection, an intrathecal injection, an intrabursal injection, an intra-tendon sheath injection, an intraperitoneal injection, an intralesional injection, a perilesional injection, and a subcutaneous injection. In one embodiment, the completely healed subject returns to pre-injury work or performance. In one embodiment, the subject is a mammal including, but not limited to human, equine, bovine, caprine, and ovine. In one embodiment, the expanded xenogen-free mesenchymal stem cell population does not contain a fetal bovine serum immunogen. In one embodiment, the expanded xenogen-free mesenchymal stem cell population does contain a bone marrow supernatant. In one embodiment, the cardiovascular injury includes, but is not limited to, a cardiomyopathy injury, a vascular abrasion injury, a myocardial infarction injury, a cardiac muscle injury

In one embodiment, the present invention contemplates a method, comprising: a) providing; i) a subject comprising a diabetes injury; and ii) a pharmaceutically acceptable composition comprising an expanded xenogen-free mesenchymal stem cell population; b) administering said pharmaceutically acceptable composition to said subject, wherein said diabetes injury is at least partially healed. In one embodiment, the diabetes injury completely heals within one year after said administering. In one embodiment, the administering comprises an injection including, but not limited to, an intra-articular injection, an intramuscular injection an extracellular tendon injection and an intravenous injection. In one embodiment, the completely healed subject returns to pre-injury work or performance. In one embodiment, the subject is a mammal that includes, but is not limited to, human, equine, bovine, caprine, and ovine. In one embodiment, the expanded xenogen-free mesenchymal stein cell population does not contain a fetal bovine serum immunogen. In one embodiment, the expanded xenogen-free mesenchymal stem cell population does contain a bone marrow supernatant. In one embodiment, the diabetes injury includes, but is not limited to a pancreatic injury, a pancreatic beta-cell injury, an ocular injury, an epithelial cell injury.

In one embodiment, the present invention contemplates a method, comprising: a) providing; i) a subject comprising a neurodegenerative injury; and ii) a pharmaceutically acceptable composition comprising an expanded xenogen-free mesenchymal stem cell population; b) administering said pharmaceutically acceptable composition to said subject, wherein said neurodegenerative injury is at least partially healed. In one embodiment, the neurodegenerative injury completely heals within one year after said administering. In one embodiment, the administering comprises an injection including, but not limited to, an intra-articular injection, an intraventricular injection, an intramuscular injection, a subcutaneous injection, and an intravenous injection. In one embodiment, the completely healed subject returns to pre-injury work or performance. In one embodiment, the subject is a mammal that includes, but is not limited to, human, equine, bovine, caprine, and ovine. In one embodiment, the expanded xenogen-free mesenchymal stem cell population does not contain a fetal bovine serum immunogen. In one embodiment, the expanded xenogen-free mesenchymal stem cell population does contain a bone marrow supernatant. In one embodiment, the neurodegenerative injury includes, but is not limited to an inflammatory neurological autoimmune condition, a primary CNS degenerative disease, Parkinson's disease (PD), Huntington's disease (HD), multiple system atrophy (MSA), amyotrophic lateral sclerosis (ALS) and Alzheimer's disease (AD).

In one embodiment, the present invention contemplates a xenogen-free cell culture medium, comprising a) a xenogen-free physiologically balanced mixture of salts; b) a xenogen-free mixture of essential amino acids; and c) a xenogen-free supplement. In one embodiment, the salt mixture and said essential amino acid mixture is a minimum essential media. In one embodiment, the xenogen-free supplement is a bone marrow supernatant. In one embodiment, the xenogen-free supplement is not a fetal bovine serum.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

The term “xenogen” or “xenogeneic” as used herein, refers to an exogenous compound, that when administered to a patient or subject, elicits an immunological response usually resulting in adverse physiological responses such as inflammation and pain.

The term “full recovery,” “healed,” or “fully healed” as used herein, refers to a patient or subject regaining an equivalent pre-injury physical capability concomitantly with an absence of symptoms associated with an injury. Such a full recovery or healing process may be monitored by parameters including, but not limited to, strength, endurance, lameness, agility, and/or pain.

The term “recovery” or “at least partially healed” as used herein, refers to a patient or subject regaining some pre-injury physical capability concomitantly with a partial absence of symptoms associated with an injury. Such a partial recovery or healing process may be monitored by parameters including, but not limited to, strength, endurance, lameness, agility, and/or pain.

The term “suspected of having”, as used herein, refers a medical condition or set of medical conditions (e.g., preliminary symptoms) exhibited by a patient that is insufficient to provide a differential diagnosis. Nonetheless, the exhibited condition(s) would justify further testing (e.g., autoantibody testing) to obtain further information on which to base a diagnosis.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀. (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent,” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “injury” as used herein, denotes a bodily disruption of the normal integrity of tissue structures. In one sense, the term is intended to encompass surgery. In another sense, the term is intended to encompass irritation, inflammation, infection, and the development of fibrosis. In another sense, the term is intended to encompass wounds including, but not limited to, contused wounds, incised wounds, lacerated wounds, non-penetrating wounds (i.e., wounds in which there is no disruption of the skin but there is injury to underlying structures), open wounds, penetrating wound, perforating wounds, puncture wounds, septic wounds, subcutaneous wounds, burn injuries etc.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients” or “subjects.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies. In veterinary practice, the term “animal” may be mammals or non-mammals including, but not limited to, equine, caprine, bovine, ovine, porcine, avian, reptilian etc.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term “immunologically active” defines the capability of a natural, recombinant or synthetic peptide, or any oligopeptide thereof, to induce a specific immune response in appropriate animals or cells and/or to bind with specific antibodies.

The term “antigenic determinant” as used herein refers to that portion of a molecule that is recognized by a particular antibody (i.e., an epitope). When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

The terms “immunogen,” “antigen,” “immunogenic” and “antigenic” refer to any substance capable of generating antibodies when introduced into an animal. By definition, an immunogen must contain at least one epitope the specific biochemical unit capable of causing an immune response), and generally contains many more. Proteins are most frequently used as immunogens, but lipid and nucleic acid moieties complexed with proteins may also act as immunogens. The latter complexes are often useful when smaller molecules with few epitopes do not stimulate a satisfactory immune response by themselves.

The term “antibody” refers to immunoglobulin evoked in animals by an immunogen (antigen). It is desired that the antibody demonstrates specificity to epitopes contained in the immunogen. The term “polyclonal antibody” refers to immunoglobulin produced from more than a single clone of plasma cells; in contrast “monoclonal antibody” refers to immunoglobulin produced from a single clone of plasma cells.

### BRIEF DESCRIPTION OF THE FIGURES

FIG. 1 presents an exemplary flow chart for bone marrow collection, processing and expansion

FIG. 2 presents an exemplary flow chart showing the biological/biochemical advantages of BMS proliferated MSCs.

FIG. 3 presents exemplary data showing a comparison in isolation and proliferation characteristics between BMS cultivated MSCs and FBS cultivated MSCs.

FIG. 3A: Colony forming units after 10 days of culture. There were significantly more CFU-Fin BMS MSCs (p=0.03) than in the FBS MSCs (BMS: median 51, range 24-209; FBS: median 33, range 15-145).

FIG. 3B: Total number of cells at the end of passage three. There was no significant difference between groups.

FIG. 3C: Population doubling time over three passages. There was no significant difference between groups.

FIG. 4 presents representative images after induction of adipogenesis. No differences were noted between MSCs cultured in BMS (left) and FBS (right).

FIG. 5 presents representative images after induction of osteogenesis. No differences were noted between MSCs cultured in BMS (left) and FBS (right).

FIG. 6 presents exemplary data showing immunomodulatory function measured by responder proliferation index after mixed lymphocyte reactions were not different between BMS and FBS MSCs.

FIG. 7 presents exemplary data showing that cell morphology was not different between representative images of MSCs cultured in BMS (left) and FBS (right) taken at the same passage and at the same time.

FIG. 8 presents exemplary data comparing immunomodulatory responses using allogeneic MSCs, autogeneic MSCs and MSCs proliferated in FBS.

FIG. 9 presents exemplary photomicrographs showing that incubating FBS proliferated MSCs in autologous serum for forty-eight (48) hours, reduced, but did not eliminate the FBS-xenogenic contamination.

FIG. 9A: MSC incubated in FBS only.

FIG. 9B: MSCs incubated in FBS followed by forty-eight (48) hour incubation in autologous serum.

FIG. 10 presents exemplary data showing FBS-xenogeneic contaminant antibodies in equine administered FBS-cultivated MSCs.

FIG. 10A: Raw data scores.

FIG. 10B: Fold-change representation of the data in FIG. 10A.

FIGS. 11A and 11B present exemplary data showing that pre-existing FBS-xenogeneic contaminant antibodies in equine serum or synovial fluid result in MSC death in culture.

FIG. 12 presents exemplary data showing that MSCs cultured with bone marrow supernatant are not destroyed by pre-existing anti-bovine antibodies.

FIG. 13 presents exemplary data showing improved clinical responses of BMS cultivated MSCs as compared to FBS cultivated MSCs.

FIG. 13A: Edema responses.

FIG. 13B: Effusion responses.

FIG. 14 presents exemplary data showing limb circumference measurements comparing BMS and FBS cultivated MSC injections into equines.

FIG. 15 presents exemplary data showing improved in vitro efficacy of BMS cultivated MSCs as compared to FBS cultivated MSCs.

FIG. 15A: Stimulation index.

FIG. 15B: Colony formation data over time.

FIG. 16 presents exemplary in vivo radiographic data showing improved cartilage injury in an injured equine within a year after intra-articular injection of MCSs cultivated in BMS.

FIGS. 17A and 17B present exemplary data showing cytokine production after one and thirty days of BMS-cultured MSCs or FBS-cultured MSCs.

FIGS. 18A and 18B presents exemplary data showing granulocyte-colony stimulating factor (G-CSF) and interferon gamm (IFN-g) production after one and thirty days of BMS-cultured MSCs or FBS-cultured MSCs.

FIG. 19 presents exemplary data of various interleukin production after one and thirty days of BMS-cultured MSCs or FBS-cultured MSCs.

FIG. 19A: IL-4 production.

FIG. 19B: IL-10 production.

FIG. 19C: IL-13 production.

FIG. 19D: IL-18 production

FIG. 20 present exemplary data showing monocyte chemoattractant protein (MCP) production after one and thirty days of BMS-cultured MSCs or FBS-cultured MSCs.

FIG. 21 summarizes cytokine production believed to most strongly mediate FBS-proliferated MSC adverse effects.

### DETAILED DESCRIPTION OF THE INVENTION

The present invention relates to therapeutic compositions of mesenchymal stem cells (MSCs). In particular, pharmaceutically acceptable MSC compositions are xenogen-free and do not have immunological adverse effects. Mesenchymal stem cells expanded in a cell culture media comprising bone marrow supernatant produce xenogen-free mesenchymal stem cells. Such xenogen-free MSC compositions improve therapy for medical conditions including, but not limited to, osteoarthritis, cardiovascular disorders and/or diabetes.

In one embodiment, the present invention contemplates a method for using bone marrow supernatant as a media additive to isolate and/or expand (e.g., proliferate) mesenchymal stem cells (MSCs). In one embodiment, the method comprises collecting bone marrow, centrifuging the bone marrow, collecting and filtering a bone marrow supernatant product, and adding the bone marrow supernatant to an MSC culture media. Although it is not necessary to understand the mechanism of an invention, it is believed that the presently disclosed bone marrow supernatant is superior to conventionally used fetal bovine serum because the bone marrow supernatant is devoid of fetal bovine serum (FBS) xenogeneic immune factors. It is further believed that such an FBS xenogeneic immune factor-free bone marrow supernatant cell culture media provides expanded MSC populations that meet or exceed US Food & Drug Administration standards for veterinary and human therapeutic compositions.

Advantages of some embodiments of this invention as compared to the conventional use of FBS in MSC culture media is the elimination of xenogens in bone marrow supernatants (BMS). Elimination of xenogeneic factors prevent immune reactions and disease transmission from bovine donors to MSC recipients. A further advantage of this invention as compared to chemically derived FBS-free products is that bone marrow supernatant is a complete biological/biochemical replacement for FBS.

The data presented herein shows the isolation and expansion of MSCs in BMS is nearly identical to MSCs in FBS in both characterization and function of MSCs. Further, MSCs proliferated in autologous BMS have been used as a therapeutic injection in twenty-four (24) equines with no adverse responses (e.g., xenogeneic immune responses).

### I. Mesenchymal Stem Cell Therapy

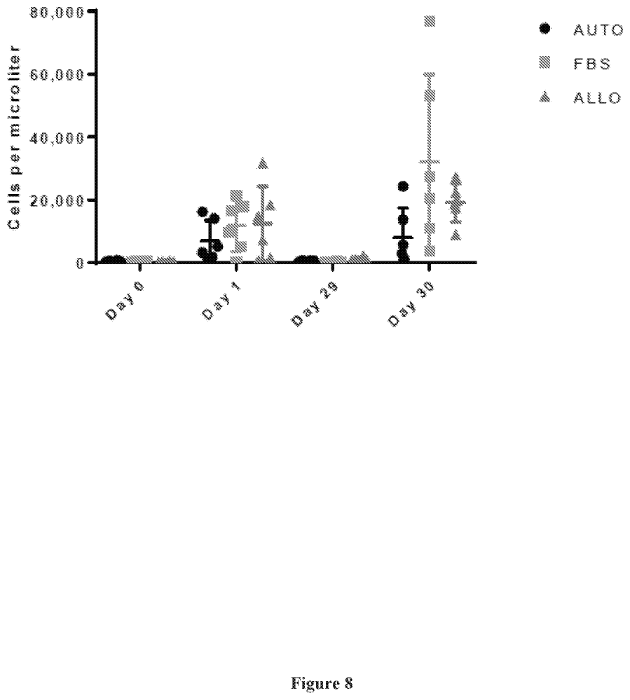

Mesenchymal stem cells or multipotent stem cells (MSCs), are currently used therapeutically for their immunomodulatory activity. For example, it has been shown that MSCs decrease T-cell proliferation and suppress cytokine production. MSCs are also thought to be anti-inflammatory mediated by TSG6 and have anabolic effects. Nonetheless, current therapies have been associated with inflammatory reactions which are suspected of being mediated by intracellular xenogen contamination. For example, xenogen contamination in FBS has been reported to cause a primed immune response. Joswig et al., “Repeated intra-articular injection of allogeneic mesenchymal stem cells causes an adverse response compared to autologous cells in the equine model”. Stem Cell Res Ther 8(1):8 (2017; see FIG. 8.

Much effort has been applied to the development of cell replacement therapies to functionally restore and replace lost or damaged tissues or organs that lack intrinsic tissue regenerative responses. Eguizabal et al., “Dedifferentiation, transdifferentiation, and reprogramming: Future directions in regenerative medicine” Semin. Reprod. Med. 31:82-94 (2013). Stem cells are a type of cell with high self-renewability and differentiation capability, which are mostly favored to be used as a candidate for cell replacement therapy. Christodoulou et al., “Comparative evaluation of human mesenchymal stem cells of fetal (Wharton's Jelly) and adult (adipose tissue) origin during prolonged in vitro expansion: Considerations for cytotherapy” Stem Cells Int. 2013:246134 (2013). In retinal degenerative diseases, research works have been focused on improving the cell recovery and regeneration of terminally-differentiated retinal neuronal cells through delivery of unmodified or modified stem cells by genetic, chemical, or mechanical manipulation Assawachananont et al., “Transplantation of embryonic and induced pluripotent stem cell-derived 3D retinal sheets into retinal degenerative mice” Stem Cell Rep. 2:662-674 (2014): Rezanejad et al., “In vitro differentiation of adipose-tissue-derived mesenchymal stem cells into neural retinal cells through expression of human PAX6 (5a) gene” Cell Tissue Res. 356:65-75 (2014); Ng et al., “Transdifferentiation of periodontal ligament-derived stem cells into retinal ganglion-like cells and its microRNA signature” Sci. Rep. 5:16429 (2015): Worthington et al., “Neuronal differentiation of induced pluripotent stein cells on surfactant templated chitosan hydrogels” Biomacromolecules 17:1684-1695 (2016).

In order to overcome the risk of stein cell rejection during allogeneic or autologous transplantation, the quest continues to focus into stem cells isolated from multipotent, adult stromal cells, such as mesenchymal stem cells (MSCs). For cells to be considered as MSCs, they phenotypically express a distinct set of cell surface markers for CD105, CD90, and CD73 but lack CD79, CD45, CD34, CD19, CD14, CD11b, and Human Leukocyte Antigen class II (HLA-II). In addition, these cells are able to undergo in vitro tri-lineage differentiation into osteogenic, adipogenic, and chondrogenic, as defined by the International Society for Cellular Therapies (ISCT) guideline for MSCs. Mok, P. L.; Leong, C. F.; Cheong, S. K. Cellular mechanisms of emerging applications of mesenchymal stem cells” Malays. J. Pathol. 35:17-32 (2013). Owing to the lack of ethical concerns related to its use, MSCs can be found abundantly in the adult tissues, such as bone marrow, adipose tissue, and dental pulp, as well as in the fetal tissues and fluids, including the umbilical cord-tissue, -blood, and -amniotic fluid and therefore lack the ethical concerns related to the collection and use of fetal stem cells. Chung et al., “Human umbilical cord blood mononuclear cells and chorionic plate-derived mesenchymal stem cells promote axon survival in a rat model of optic nerve crush injury” Int. J. Mol. Med. 37:1170-1180 (2016); Roth et al., “Hypoxic-Preconditioned bone marrow stem cell medium significantly improves outcome after retinal ischemia in rats” Investig. Ophthalmol. Vis. Sci. 57:3522-3532 (2016); Ezquer et al., “Intravitreal administration of multipotent mesenchymal stromal cells triggers a cytoprotective microenvironment in the retina of diabetic mice” Stem Cell Res. Ther. 7: 42 (2016); Leow et al., “Safety and efficacy of human Wharton's Jelly-derived mesenchymal stem cells therapy for retinal degeneration” PLoS ONE 10: e0128973 (2015); Mead et al., “Mesenchymal stromal cell-mediated neuroprotection and functional preservation of retinal ganglion cells in a rodent model of glaucoma” Cytotherapy 18:487-496 (2016); and Kim et al., “Retinal angiogenesis effects of TGF-1 and paracrine factors secreted from human placental stem cells in response to a pathological environment” Cell Transplant. 25:1145-1157 (2016).

### II. Conventional Mesenchymal Stem Cell Cultures

Mesenchymal stem cells (MSCs) are routinely expanded using fetal bovine serum (FBS). Centeno et al., “Bone Marrow Adipose Portion Isolation Device And Methods”, U.S. Pat. No. 9,976,115 (herein incorporated by reference). The presence of xenogen in stem cell media is believed to cause a negative inflammatory reaction due intracellular bovine protein at the time of therapeutic MSC injection. Platelet lysates and releasates have failed to reliably produce an MSC that is unchanged from FBS supplemented MSCs. In order to eliminate adverse reactions to xenogen and to minimize risk of disease transmission, the art was challenged to find an alternative to FBS in the culture of MSCs to provide a pharmaceutically acceptable therapeutic.

One report used a cell culture media that contained both FBS and BMS to culture MSCs. Bostock et al., “The Use Of Bone Marrow Supernatant In 2D And 3D Mesenchymal Stem Cell Culture” ORS, Poster 1549 (2012). While some improvement in MSC proliferation was observed, the data presented herein predicts that adverse effects would still be present when therapeutically administered because of residual FBS xenogens in the final expanded MSC population.

FBS-depleted MSC cultures were reported to improve the adverse effects of intra-articular injection into equines. However, this protocol first incubated the MSCs in fluorescently labeled FBS to initiate expansion followed by a forty-eight (48) hour incubation in standard cell culture media. FBS depletion was monitored by a loss in fluorescence in the MSC culture media. Joswig et al. (supra). While the data in Joswig et al. shows a significant trend of lameness improvement over a thirty (30) day period, these data do not predict a complete recovery (e.g., 100% of treated equines) during the subsequent year as attained by embodiments of the present invention (infra). Although it is not necessary to understand the mechanism of an invention, it is believed that the FBS “wash-out” left significant xenogeneic immunogens that were not detectable using the fluorescently-tagged FBS. In fact, Joswig et al. discloses that a direct measurement of the FBS-free MSC cultures was only approximately 95% xenogen-free. One of skill in the art might expect this residual 5% xenogen contamination to have a clinical effect. This expectation was substantiated in a follow-up evaluation of subjects treated in Joswig et al, as well as subsequent subjects that were treated in accordance with Joswig et al.. Ninety-seven (97) total subjects (e.g., horses) were treated with FBS-depleted auto-MSC cultures by washing with BMS for the last 24-48 hours of proliferation. Of these ninety-seven subjects, twenty-five (25) were joint injuries (e.g, osteoarthritis) and seventy-two (72) were tendon/soft tissue injuries. Of those cases, the overall clinical assessment was that there was mixed efficacy with a moderate clinical response on average (data not shown).

### III. Bone Marrow Supernatant Mesenchymal Stem Cell Cultures

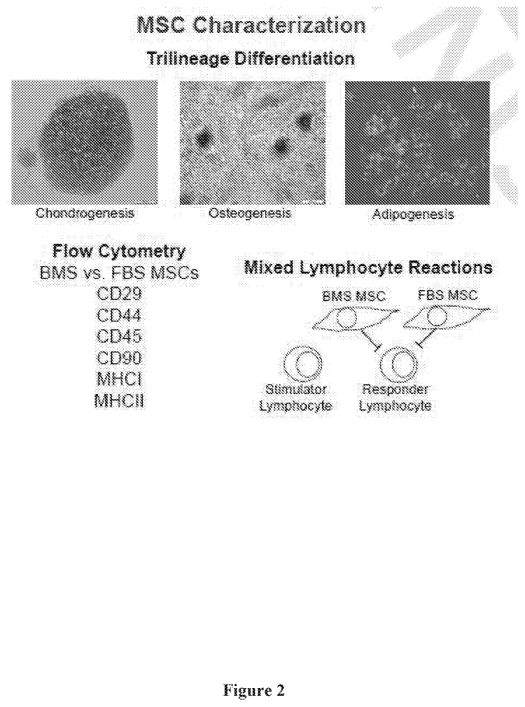

In one embodiment, the present invention contemplates a method for producing MSCs in BMS-supplemented cultures that differ from conventional FBS-supplemented cultures in parameters including, but not limited to, isolation characteristics, expansion characteristics, cell surface protein markers and trilineage differentiation characteristics (e.g., such as performance in mixed lymphocyte reaction). See, FIG. 2.

The use of bone marrow supernatant has been reported to be useful as an injection adjuvant for proliferated MSCs, but fetal bovine serum was included in the MSC-expansion culture media. Smith et al., “Pharmaceutical Kits Comprising Mesenchymal Stem Cells” U.S. Pat. No. 8,178,084 (herein incorporated by reference).

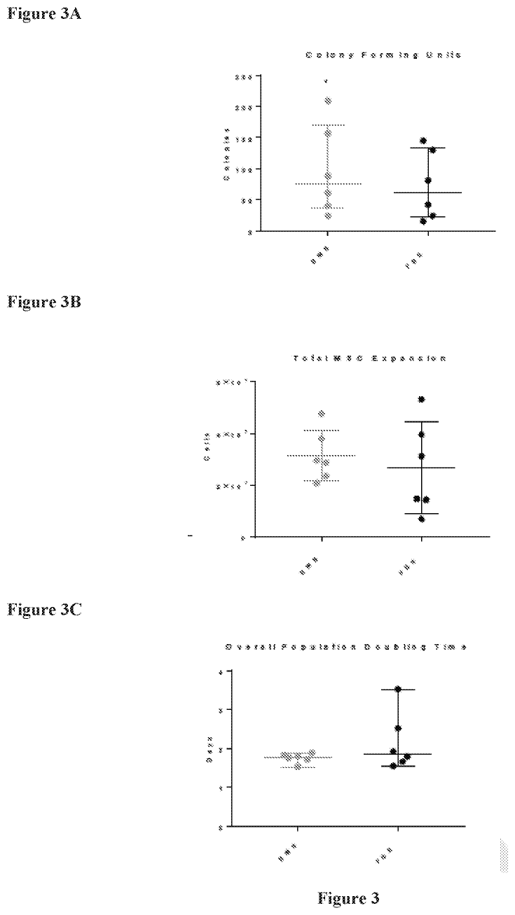

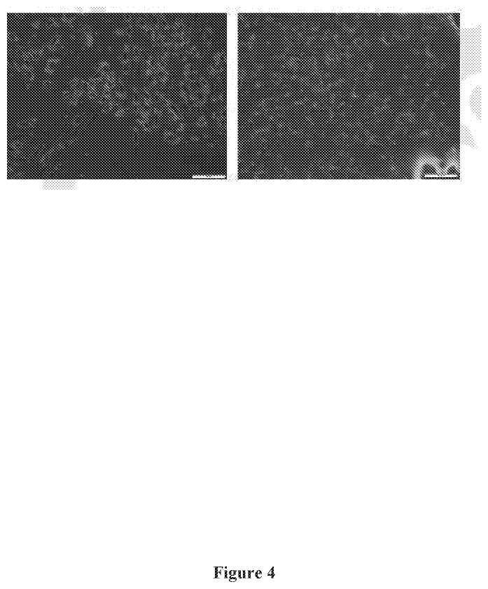

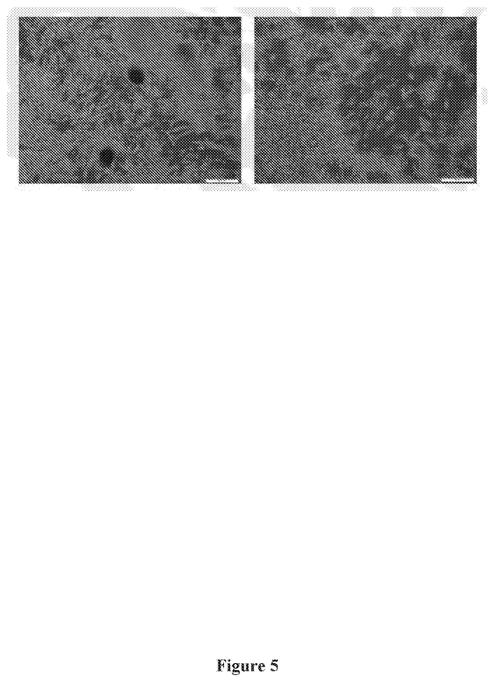

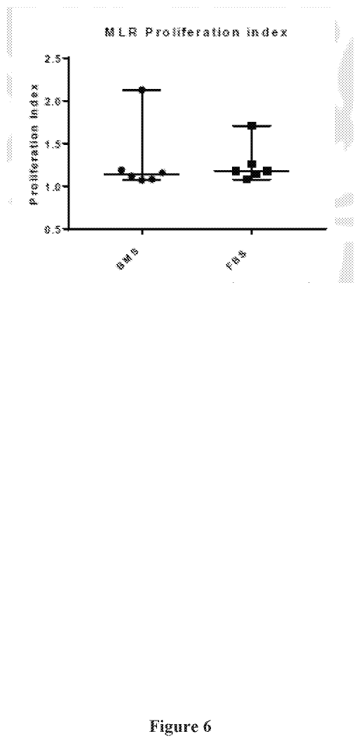

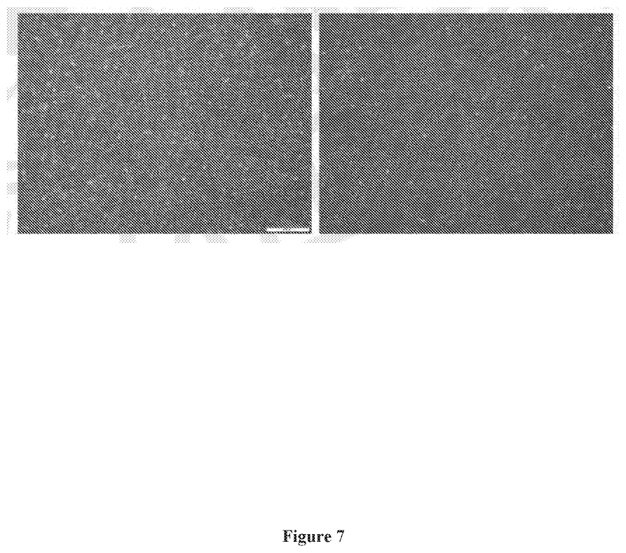

The characteristics of MSCs proliferated in BMS-containing media were compared to MSCs proliferated in FBS-containing media. The data show that MSCs were isolated in greater quantity when proliferated in BMS versus FBS, but there was no apparent differences in post-passage cell number and cell population doubling times. See, FIGS. 3A-C. Further characterization between BMS- and FBS-cultivated cells showed that there were no differences in: i) apidogenesis (See, FIG. 4); ii) osteogenesis (See, FIG. 5), iii) mixed lymphocyte reactions (See, FIG. 6); iv) morphology (See, FIG. 7). Specific cell surface protein markers were also assayed where no differences were identified. See, Table 1.

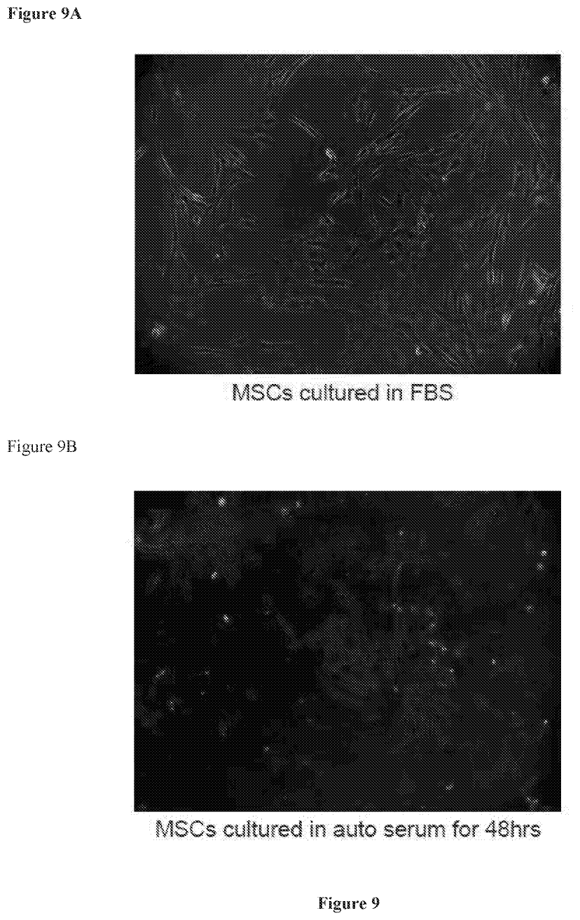

These data suggest that BMS appears to be a suitable alternative to FBS in the isolation of equine bone marrow derived mesenchynmal stem cells. Although it is not necessary to understand the mechanism of an invention, it is believed that the use of BMS, rather than PBS, eliminates xenogen contamination in an MSC culture and the associated risk of an adverse inflammatory response after administration. It was observed that incubating FBS proliferated MSCs in autologous serum for forty-eight (48) hours, reduced, but did not eliminate the FBS-xenogenic contamination. See, FIG. 9.

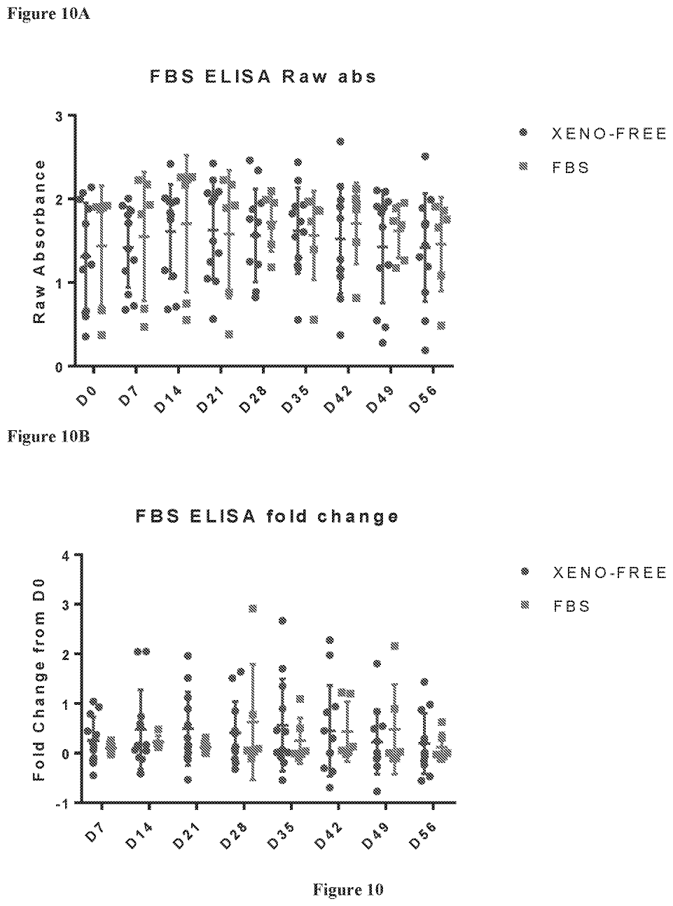

A measurable level of antibodies to FBS was observed in all equines administered MSCs cultivated in FBS. See, FIG. 10A. When looking at the fold-change, there is a slight trend that the PBS group increases over time, but not significantly. See, FIG. 10B.

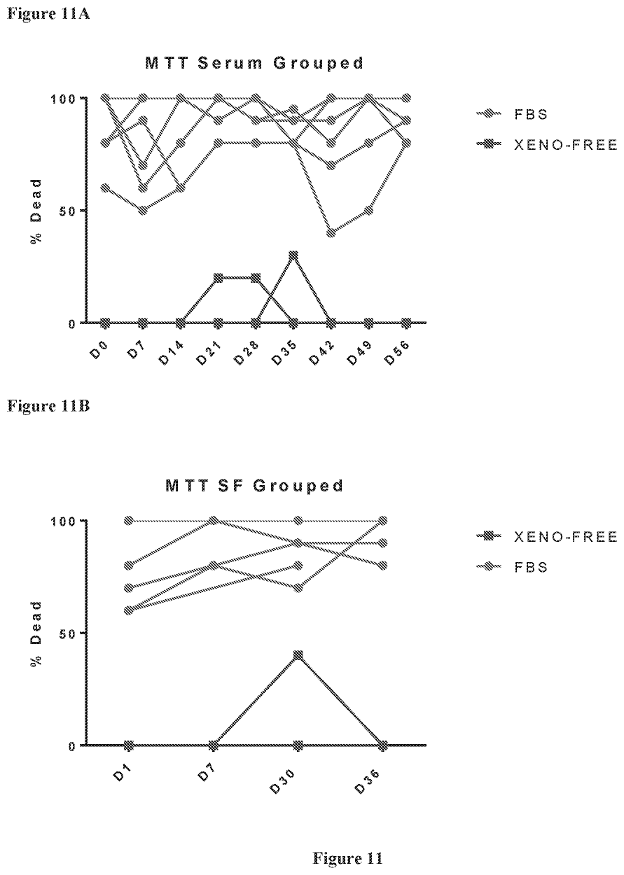

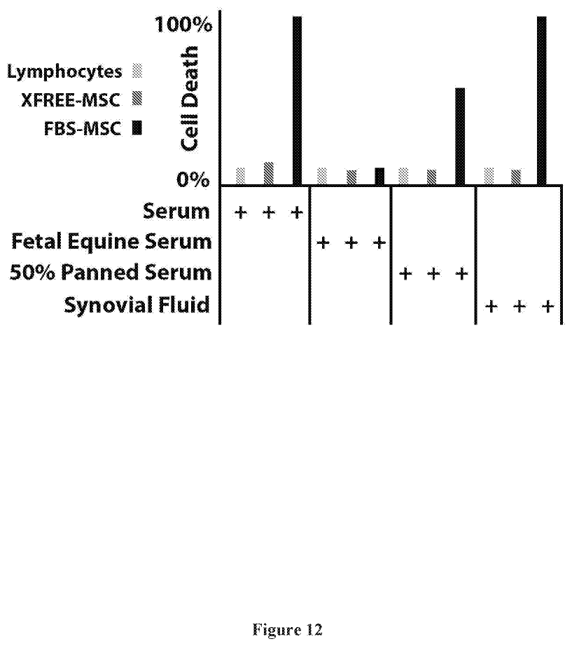

Microcytotoxicity assays are a measure of cell death due to preformed antibodies in serum and synovial fluid against antigens presented by MHCI. Routinely vaccinated horses have anti-bovine antibodies because of bovine contaminants in vaccines. MSC cell death was measured after incubation with autologous serum. The data shows that the horses have antibodies to FBS prior to injection, and that these pre-existing antibodies are sufficient to kill MSCs grown in FBS in both serum and synovial fluid. See, FIGS. 11A & 11B. This preliminary data was complemented with a direct comparison of FBS and BMS cultivated MSC death in culture. When serum or synovial fluid from vaccinated equines are added to MSCs in culture, there is marked cell death when MSCs are cultured with fetal bovine serum (FBS-MSCs), and negligible cell death noted when MSCs are cultured with bone marrow supernatant (BMS-MSCs). See, FIG. 12. When MSCs are combined with panned serum, having reduced bovine antibodies by 50%, there is reduction in cell death of FBS-MSCs. When MSCs are combined with fetal equine serum, which is devoid of anti-bovine antibodies, there is also negligible cell death in all cell types. See, FIG. 12, This demonstrates that in vivo MSCs cultured with FBS will be targeted for immune destruction, thus precluding MSC efficacy.

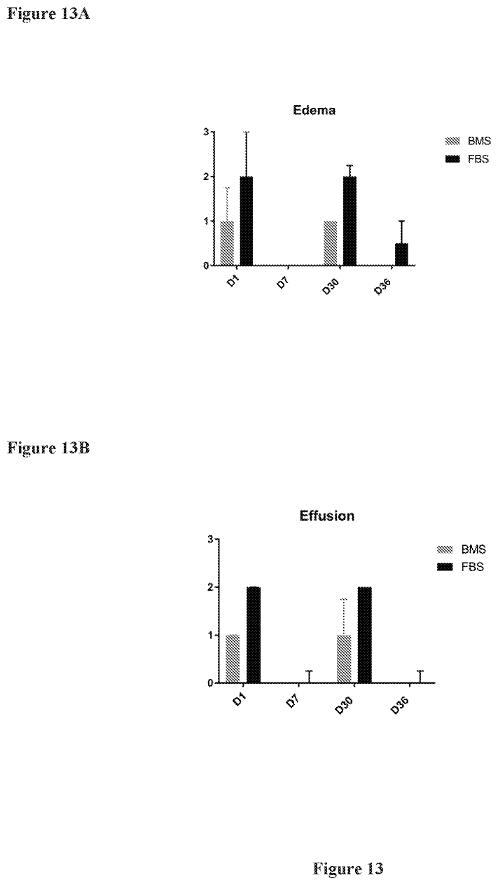

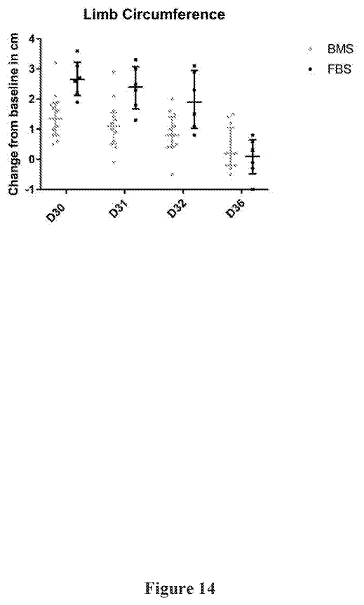

In one embodiment, the present invention contemplates a method comprising determining a clinical response to BMS MSCs. In one embodiment, the clinical response comprises an improved longevity of BMS MSCs therapeutic efficacy as compared to FBS MSCs. For example, in vivo data demonstrates there is less adverse reaction after a therapeutic administration with BMS-MSCs compared to FBS-MSCs. The data presented herein demonstrates that both edema and effusion are significantly reduced with the administration of BMS cultivated MSCs as compared to FBS cultivated MSCs to equines. Subjective scores were determined for edema and effusion present after injection of MSCs cultured in BMS or FBS on days 0 and 29. Both are a measure of adverse clinical reaction, with greater edema and effusion being a sign of inflammation. Both are significantly decreased with BMS-MSCs compared to FBS-MSCs. See, FIGS. 13A and 13B. Limb circumference measurements corrected to baseline (prior to injection) after second injection with MSCs were additionally determined on day 29. Limb circumference is an objective measure of both edema and effusion present and confirms the above direct measurements. The data show significantly less increase in limb circumference with BMS-MSCs compared to FBS-MSCs. See, FIG. 14.

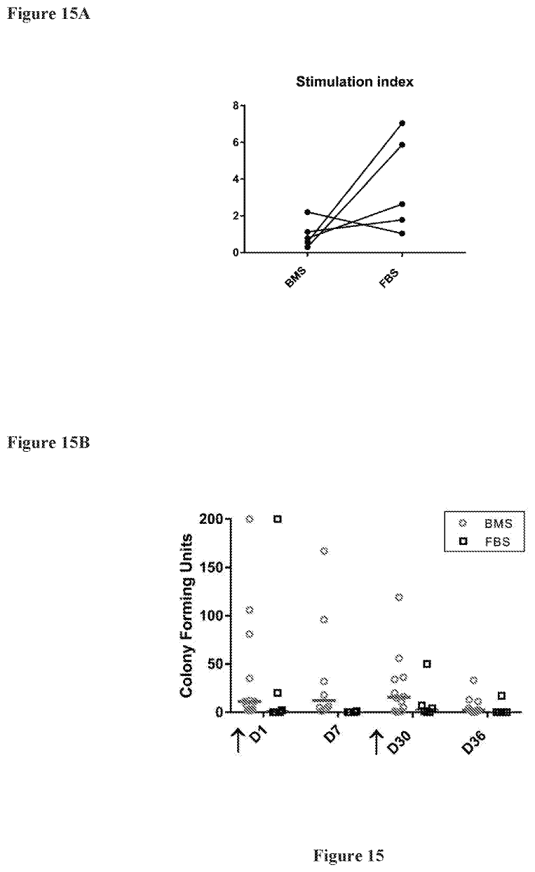

In one embodiment, the present invention contemplates a method comprising BMS cultivated MSCs having improved efficacy as compared to FBS cultivated MSCs. For example, in vitro assays were performed to compare such efficacies. Mixed lymphocyte reactions were used to measure a stimulation index, which indicates immunomodulation by MSCs in vitro. Stimulation index is the outcome parameter of mixed lymphocyte reactions and a measure of MSC efficacy. A lower stimulation index indicates improved ability of an MSC to suppress naturally occurring inflammation. Points represent MSCs from a single horse, with lines connecting points from one horse in different growth conditions. The data suggest a trend of less lymphocyte stimulation in BMS supplemented MSCs as compared to FBS supplemented MSCs. See, FIG. 15A. In vivo colony forming unit assay to measure the presence of synovial MSCs. Autologous MSCs were injected on days 0 and 29 (black arrows), and synovial fluid retrieved on days 1, 7, 30, and 36. Increased colonies isolated in the BMS-MSC group indicate enhanced recruitment of endogenous MSC progenitors. This measure of MSC efficacy shows improved function of BMS-MSCs compared to FBS-MSCs. See, FIG. 15B.

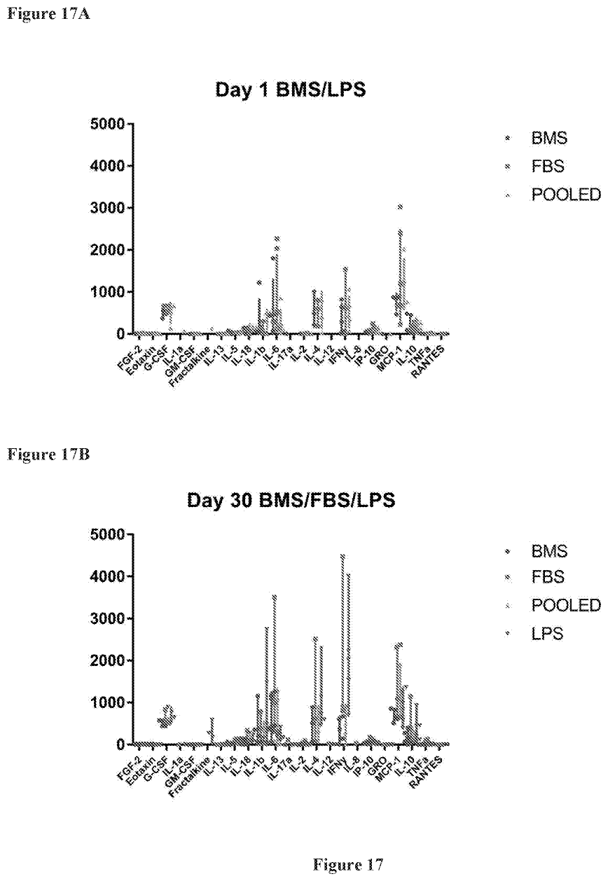

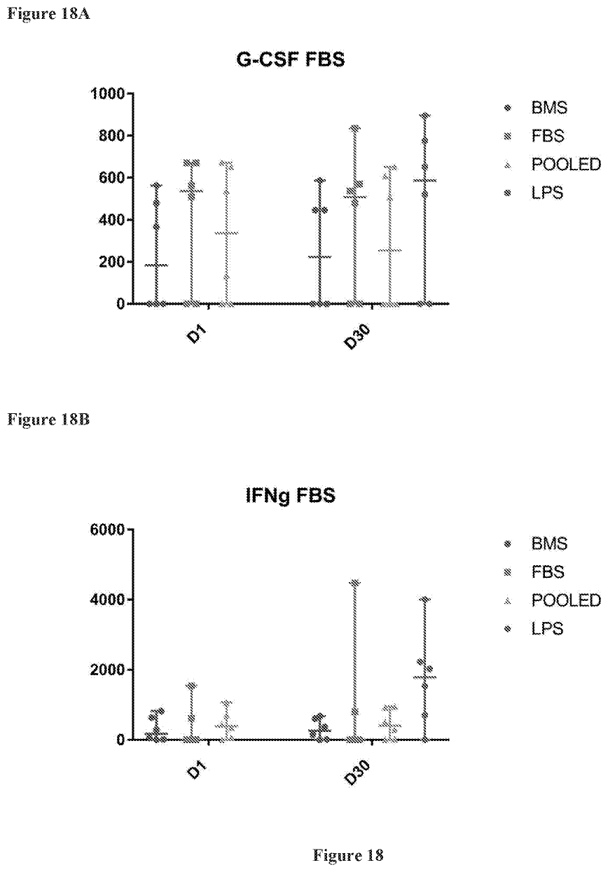

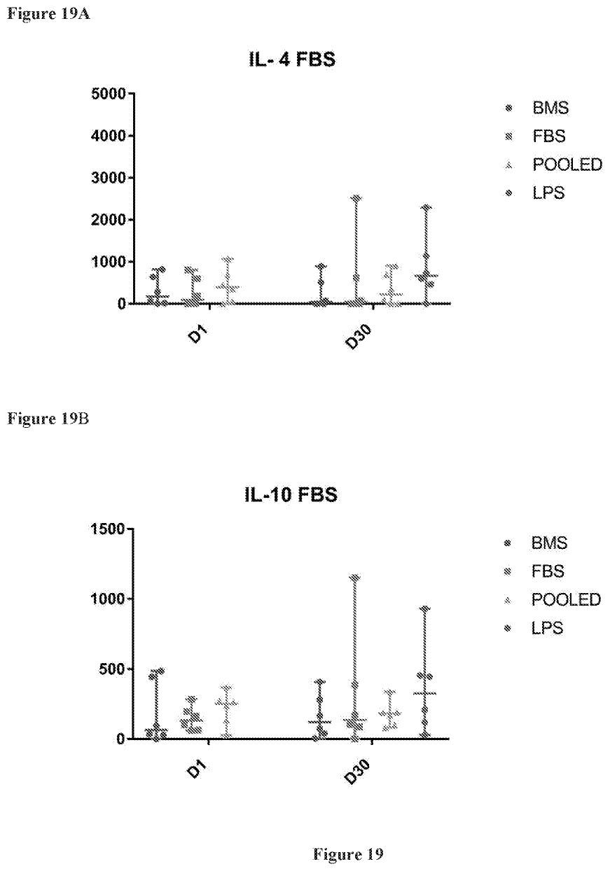

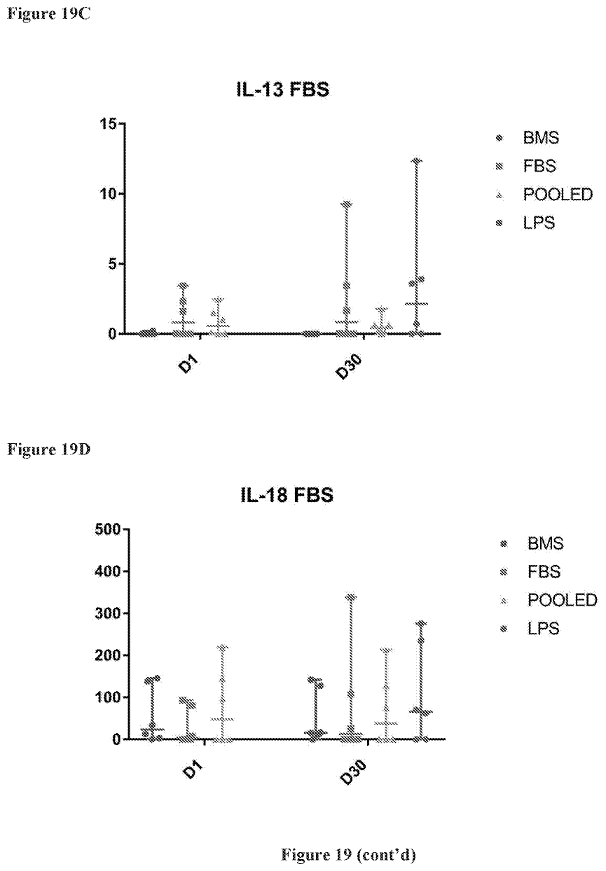

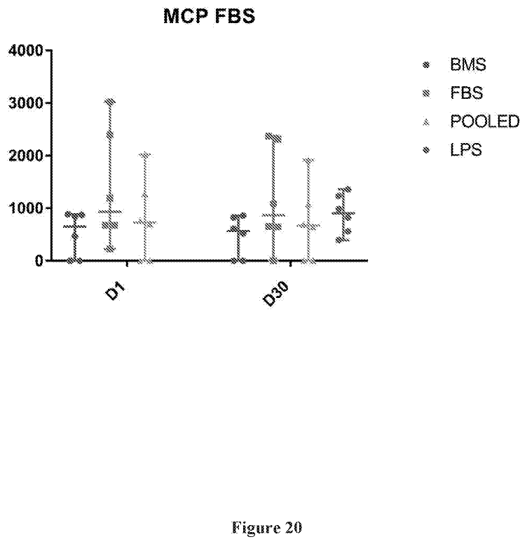

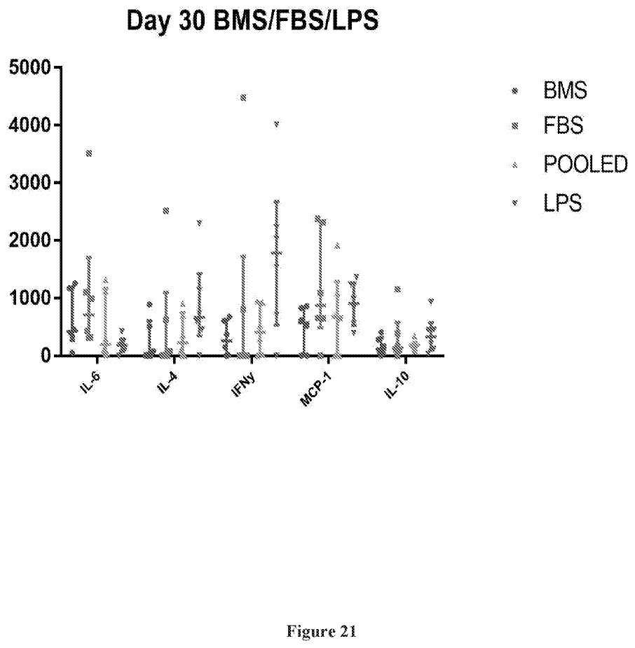

Production of numerous cytokines was assayed in comparison between BMS proliferated MSCs and FBS proliferated MSCs. The data show that cytokine production was consistently higher in all cytokines in the FBS-proliferated MSCs as opposed to the BMS-proliferated MSCs. See, FIGS. 17A and 17B. A close comparison of specific cytokine production and their response to liposaccharide (LPS) stimulation was then performed, again showing that the FBS-proliferated MSCs generated a significantly greater amount of cytokines than the BMS-proliferated MSCs; i) granulocyte-colony stimulating factor (FIG. 18A); ii) interferon gamma (FIG. 18B); iii) interleukin-4 (FIG. 19A); iv) interleukin-10 (FIG. 19B); v) interleukin-13 (FIG. 19C); vi) interleukin-18 (FIG. 19D); and vi) monocyte chemoattractant protein-1. (FIG. 20). For clarity, cytokine production believed to most strongly mediate FBS-proliferated MSC adverse effects is summarized for data collected after thirty days (e.g., IL-6, IL-4, IFN-g, MCP-1 and IL-10). See, FIG. 21.

### IV. Treatment of Inflammatory Conditions With Xenogen-Free MSCs

In one embodiment, the present invention contemplates the treatment of a subject exhibiting at least one symptom of an inflammatory medical condition with a composition comprising a xenogen-free MSC. In one embodiment, the inflammatory medical condition comprises a musculoskelatal injury. In one embodiment, the inflammatory medical condition comprises a cardiovascular injury. In one embodiment, the inflammatory medical condition comprises a diabetes injury.

In addition to their wide distribution, MSCs are also known to possess minimal susceptibility to malignant transformation and are capable of avoiding immune cell recognition, hence providing a potential platform for allogeneic and autologous cell transplants. Zhao et al., “Mesenchymal stem cells: Immunomodulatory capability and clinical potential in immune diseases” J. Cell. Immunother. 2:3-20 (2016). Collectively, MSCs have been widely employed in various acute and chronic neurodegenerative conditions, including central-peripheral neuropathy, stroke, spinal cord injury, as well as ocular degenerative disorders. Cejka et al., “The favorable effect of mesenchymal stem cell treatment on the antioxidant protective mechanism in the corneal epithelium and renewal of corneal optical properties changed after alkali burns” Oxid. Med. Cell. Longev. 2016:5843809 (2016); Schafer et al., “Mesenchymal stem/stromal cells in regenerative medicine: Can preconditioning strategies improve therapeutic efficacy?” Transfus. Med. Hemother. 43:256-267 (2016); Zeng et al., “Autocrine fibronectin from differentiating mesenchymal stem cells induces the neurite elongation in vitro and promotes nerve fiber regeneration in transected spinal cord injury” J. Biomed. Mater. Res. A 104:1902-1911 (2016); Nakano et al., “Bone marrow-derived mesenchymal stem cells improve diabetes-induced cognitive impairment by exosome transfer into damaged neurons and astrocytes” Sci. Rep. 6:24805 (2016).

A. Musculoskeletal Injury Treatment

Mesenchymal stromal cells (MSCs) have firmly occupied the attention of orthopedic clinicians and scientists for most of the last 25 years. Hundreds of laboratories world-wide have carried out research aimed at unraveling the biological characteristics of these cells and probing the manner in which they potentially contribute to cartilage and bone repair. Clinical trials registries indicate that they are also being tested in patient studies for a wide range of conditions such as osteoarthritis, rheumatoid arthritis, fracture repair, regeneration of articular cartilage, tendon repair, and for treatment of degenerative disc disease. Despite these efforts, the effectiveness of MSCs as a treatment modality for these conditions is still uncertain and market authorizations have been limited. In addition, critical and clear phenotypic parameters for defining MSCs are uncertain and a coherent biological framework surrounding the therapeutic mechanism of action is not yet available. Added to this, cell manufacturing protocols are complex and costly and present substantial challenges in terms of regulatory oversight and standardization. Despite these obstacles, MSCs still remain at the forefront of efforts in Regenerative Medicine, based on a conviction that this technology can provide an effective treatment paradigm for major diseases where there is still an unmet need. Barry et al., “MSC Therapy for Osteoarthritis: An Unfinished Story” J Orthop Res 37:1229-1235 (2019).

MSCs are being tested in a wide array of clinical conditions based on the premise that they can provide broad acting regenerative, immunomodulatory, or antiinflammatory activity. One of the striking aspects, evidenced in an analysis of clinical trial registries, is the number of different indications that are being studied, ranging from autoimmune conditions, neurological diseases, complications of diabetes, cardiomyopathy, retinal diseases as well as orthopedic indications. For many of these, the biological basis is uncertain but the explosion in clinical interest in MSC therapies has given rise to an idea that these cells represent a therapeutic panacea. Tyndall A., “Mesenchymal stem cell treatments in rheumatology: a glass half full?” Nat Rev Rheumatol 10:117-124 (2014).

It has been reported that MSCs do indeed have a regenerative effect in joint disease, specifically showing injury repair when MSCs were delivered to a knee joint afflicted with osteoarthritis (OA). MSCs were delivered as a suspension by intra-articular injection and diminishes many degenerative changes. Erosion of the articular cartilage, osteophyte formation, and subchondral sclerosis, all symptoms of advanced OA, were significantly less evident in the cell—treated knees compared with those treated with vehicle only. This was significant because it showed that intra-articular injection was more effective than scaffold placement. The cells, delivered directly to the synovial fluid compartment, were capable of untargeted attachment. Further experiments tracing the engraftment of labeled cells gave rise to two key observations: (i) the retention of cells in the OA joint was very low, typically around 3%, indicating that the majority of cells disappeared within a few days, and (ii) only a tiny portion of the retained cells were attached to the cartilage surface. The majority of the retained cells were associated with the synovium, meniscus, and fat pad. The interpretation of these experiments was reasonably clear-MSCs did not act as cell replacement therapy and exerted their effects by an alternate mechanism. The alternate mechanism that was widely suggested was a paracrine effect, whereby MSCs secrete, and deliver to the host, an array of repair mediators, growth factors, cytokines, and other molecules that stimulate host cells to mount a repair response. Murphy et al., “Stem cell therapy in a caprine model of osteoarthritis” Arthritis Rheum 48:3464-3474 (2003).

There has been a great deal of investigation into the use of MSCs as a treatment for OA. This effort has been justified because OA is still a major unaddressed disease with an exceptionally high global burden. With aging populations and a rise in obesity this burden will increase. It is interesting to note that, of all surgical procedures carried out in hospitals in the United States, those which treat patients with arthritic disease are amongst the most common and most costly. The direct and indirect economic costs associated with OA are immense and a new therapeutic approach, demonstrating disease-modifying activity, will be of substantial benefit. There are many published studies describing the clinical assessment of MSCs in the treatment of OA. In assessing these, it is difficult to find a consistent approach that will lead to an unambiguous interpretation. Most published papers relate to relatively small patient numbers and an almost dizzying array of approaches in terms of trial design, the tissue source of the cells and the protocols used to isolate them, the lack of standardized and reliable phenotypic analysis and the inclusion of additional biological components for delivery, such as platelet lysate, platelet-rich plasma, hyaluronan, and so on. This inconsistent and variable approach has led some authors to suggest that most of the published clinical trials that were conducted to assess the effectiveness of MSC treatment for OA are unlikely to contribute to an evidence-based understanding. Piuzzi et al., “Proceedings of the signature series symposium “cellular therapies for orthopaedics and musculoskeletal disease proven and unproven therapies—promise, facts and fantasy,” International Society For Cellular Therapies, Montreal, Canada, May 2, 2018” Cytotherapy 20:1381-1400 (2018).

In one clinical trial review, 61 studies which treated 2,390 patients with OA was evaluated. The objective was to identify those trials which presented a high level of evidence in the form of a randomized study or prospective patient cohort and distinguish these from studies which presented a lower level of evidence, such as a retrospective cohort or case study. Their assessment was that only 14 of the studies, where a total of 288 patients were treated, could be entered into the evidence database. Jevotovsky et al., “Osteoarthritis and stein cell therapy in humans: a systematic review” Osteoarthritis Cartilage 26:711-729 (2018)

Clearly, this does not provide an unambiguous or definitive conclusion regarding efficacy. Phase 3 studies are needed, with rigorous attention to design where the trials are fully blinded, randomized, placebo-controlled, and multi-entered and where a sufficiently large number of patients is enrolled with a long-term follow-up. It is also clear that outcome measures should be selected to distinguish between symptom-modifying and disease-modifying effects. This is because the complexity, risk, and high cost of cellular therapies make it imperative that there is a quantifiable and significant disease-modifying outcome, so structural analysis of repair in the form of magnetic resonance imaging and radiographic assessment is necessary. Despite the shortcomings of the clinical database, the available assessments suggest that MSC therapy is likely to be effective and to provide a new treatment paradigm where none exists currently. Barry et al., “MSC Therapy for Osteoarthritis: An Unfinished Story” J Orthop Res 37:1229-1235 (2019).

A phase I dose escalation study has been reported regarding an adipose-derived MSCs (ASCs) treatment for severe OA of the knee. In this study, 18 patients with symptomatic, severe knee OA were treated with a single intra-articular injection of autologous ASCs. The treatment consisted of a single intra-articular injection of 2×10⁶, 10×10⁶, or 50×10⁶ cells (n=6) and the patients were followed up for 6 months after treatment. The results indicated that the treatment was safe, with no serious adverse events reported. The study, although lacking statistical significance, gave rise to two interesting observations: (i) there was an improvement in pain outcome within 24 h of treatment that was sustained during the follow-up period and (ii) patients who received the lowest dose showed the greatest improvement compared with baseline. Pers et al., “Adipose mesenchymal stromal cell—based therapy for severe osteoarthritis of the knee: a phase I dose—escalation trial” Stem Cells Transl Med 5:847-856 (2016).

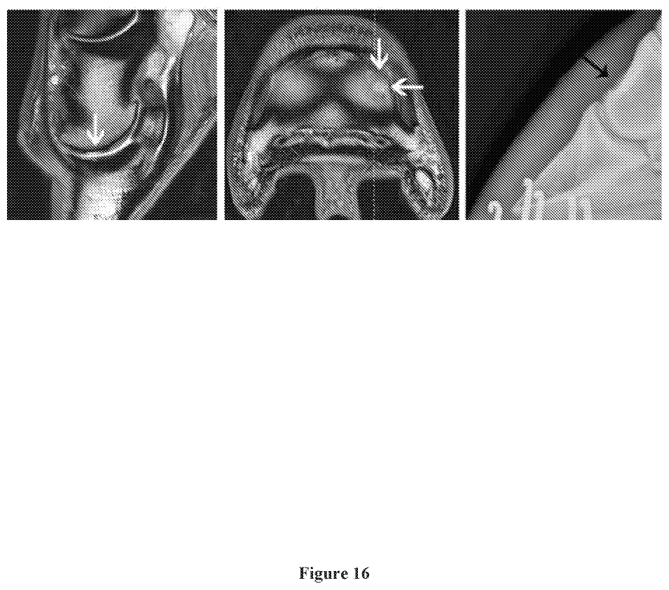

The data presented herein demonstrated an improved in vivo therapeutic efficacy comparing BMS cultivated MCSs and FBS cultivated MSCs using radiographic imaging. Radiographic articular cartilage injury (white arrows) and osteophytes (black arrow) in the coffin joint of a 13-yo Quarter Horse gelding. Given the severity of cartilage damage, this was considered a career ending injury at the time of diagnosis and chronic lameness was the expected outcome. The joint received four injections of BMS-MSCs. The lameness resolved and he returned to performance (12 months follow-up). See, FIG. 16. Twelve (12) horses with naturally occurring musculoskeletal injuries were also treated with BMS cultivated MSCs. The data show a marked improvement in lameness and performance where all treated horses made complete recoveries. See, FIG. 2.

TABLE 2
Representative Data Showing In Vivo
Equine Injury Recovery With BMS-MCSs

| Location | Number of | Average Number | Return to |
|---|---|---|---|
| Treated | Patients | of Injections | Work |
| Coffin joint | 3 | 4 | 3/3 |
| Carpus (knee) | 2 | 3 | 2/2 |
| Elbow | 1 | 3 | 1/1 |
| Tendon sheath | 2 | 1 | 2/2 |
| Fetlock | 1 | 2 | 1/1 |
| Tendon injury | 3 | 2 | 3/3 |

Complete injury healing for osteoarthritic conditions (e.g., a coffin joint injury) has never been reported using FBS cultivated MSCs.

B. Cardiovascular Injury Treatment

Several previous reports demonstrate, in large animal models, major degrees of infarct size reduction and functional recovery with MSC cell therapy. Amado et al., “Cardiac repair with intramyocardial injection of allogeneic mesenchymal stem cells after myocardial infarction” Proc Natl Acad Sci USA. 102:11474-11479 (2005); Quevedo et al., “Allogeneic mesenchymal stem cells restore cardiac function in chronic ischemic cardiomyopathy via trilineage differentiating capacity” Proc Natl Acad Sci USA. 106:14022-14027 (2009); Amado et al., “Multimodality noninvasive imaging demonstrates in vivo cardiac regeneration after mesenchymal stem cell therapy” J Am Coll Cardiol. 48:2116-2124 (2006); Schuleri et al., “Early improvement in cardiac tissue perfusion due to mesenchymal stem cells” Am J Physiol Heart Circ Physiol. 294:1H2002-H2011 (2008) Schuleri et al., “Autologous mesenchymal stem cells produce reverse remodelling in chronic ischaemic cardiomyopathy” Eur Heart J. 30:2722-2732 (2009); Silva et al., “Mesenchymal stem cells differentiate into an endothelial phenotype, enhance vascular density, and improve heart function in a canine chronic ischemia model” Circulation 111:150-156 (2005). Lim et al., “The effects of mesenchymal stem cells transduced with Akt in a porcine myocardial infarction model” Cardiovasc Res. 70:530-542 (2006); Freyman et al, “A quantitative, randomized study evaluating three methods of mesenchymal stem cell delivery following myocardial infarction” Eur Heart J. 27:1114-1122 (2006).

The regenerative potential of the heart is insufficient to fully restore functioning myocardium after injury, motivating the quest for a cell-based replacement strategy. Bone marrow derived mesenchymal stem cells (MSC) have the capacity for cardiac repair that appears to exceed their capacity for differentiation into cardiac myocytes. Bone marrow derived MSCs can stimulate the proliferation and differentiation of endogenous cardiac stem cells (CSCs) as part of their regenerative repertoire. For example, it was been reported that Female Yorkshire pigs (n=31) underwent experimental myocardial Infarction (MI); and 3 days later received transendocardial injections of allogeneic male bone marrow-derived MSCs, MSC concentrated conditioned medium (CCM), or placebo (Plasmalyte). A no-injection control group was also studied. MSCs engrafted and differentiated into cardiomyocytes and vascular structures. In addition, endogenous c-kit+ CSCs increased 20-fold in MSC treated animals vs. controls (p<0.001), there was a 6-fold increase in GATA-4+ CSCs in MSC vs. control (p<0.001), and mitotic myocytes increased 4-fold. Porcine endomyocardial biopsies were harvested and plated as organotypic cultures in the presence or absence of MSC feeder layers. In vitro, MSCs stimulated c-kit+ CSCs proliferation into enriched populations of adult cardioblasts that expressed Nkx2-5 and troponin I. Hatzistergos et al., “Bone marrow mesenchymal stem cells stimulate cardiac stem cell proliferation and differentiation” Circ Res. 107(7):913-922 (2010). At approximately 50% confluency the media was removed and replaced with 5 ml of transduction media consisting of alpha MEM plus 20% FCS plus 8 ug/ml polybrene and 10 ul of lentiviral vector LV-173GFP (Lentigen, Gaithersburg, Md.).

It has previously been reported that the reappearance of myocardial tissue in the border zones of infarction including a rim of tissue on the endocardial surface is associated with improved tissue perfusion and recovery of regional function. Amado et al., “Cardiac repair with intramyocardial injection of allogeneic mesenchymal stem cells after myocardial infarction” Proc Natl Acad Sci USA. 102:11474-11479 (2005); Amado et al., “Multimodality noninvasive imaging demonstrates in vivo cardiac regeneration after mesenchymal stem cell therapy” J Am Coll Cardiol. 48:2116-2124 (2006); Schuleri et al., “Early improvement in cardiac tissue perfusion due to mesenchymal stem cells” Am J Physiol Heart Circ Physiol. 294:H2002-H2011 (2008). Despite the reproducible demonstration of major cardiac recovery with MSC therapy, the underlying mechanism of action has been a challenge to demonstrate given the rarity of differentiated MSC-derived myocytes in the post infarction heart. For example, this effect may include MSC engraftment and trilineage differentiation. MSCs also interact with host CPCs, promoting their recruitment and/or expansion and differentiation. In addition, there is evidence of extensive myocyte mitosis which very likely represents the terminal stage of cycling of CPC derived myocytes. Kajstura et al., “Cardiomyogenesis in the Adult Human Heart” Circ Res. (2010).

The use of MSC in different models of cerebrovascular diseases, especially stroke, has been documented in various studies in several models. One study demonstrated that MSC transplantation has the potential to repair the ischemia-damaged neural networks and restor lost neuronal connection. The recovered circuit activity contributed to the improved sensory motor function post-transplantation. In a model of intracerebral hemorrhage in rats, human MSC (derived from adipose tissue) were transplanted via femoral iv. administration. The study demonstrated that the transplanted cells were detectable at the injured tissue. Functionally, the treated animals showed impressive improvement as evaluated by behavioral tests. Chen et al., “Intravenous bone marrow stromal cell therapy reduces apoptosis and promotes endogenous cell proliferation after stroke in female rat” J. Neurosci. Res. 73(6):778-786 (2003); Andres et al., “Potential of adult neural stem cells in stroke therapy” Regen. Med. 3(6):893-905 (2008); Gutierrez-Fernandez et al., “Functional recovery after hematic administration of allogenic mesenchymal stem cells in acute ischemic stroke in rats” Neuroscience 175:394-405 (2011)” Yoo et al., “Mesenchymal stem cells promote proliferation of endogenous neural stem cells and survival of newborn cells in a rat stroke model” Exp. Mol. Med. 40(4):387-397 (2008): Song et al., “Restoration of intracortical and thalamocortical circuits after transplantation of bone marrow mesenchymal stem cells into the ischemic brain of mice” Cell Transplant. Epub (2012); Yang et al., “Human adipose-derived stein cells for the treatment of intracerebral hemorrhage in rats via femoral intravenous injection” Cell Mol. Biol. Lett. 17(3):376-392 (2012).

Ischemic cardiomyopathy (IC M) and dilated cardiomyopathy (DCM) differ in histopathology and prognosis. Although transendocardial delivery of mesenchymal stem cells is safe and provides cardiovascular benefits in both, a comparison of mesenchymal stem cell efficacy in ICM versus DCM has not been done. A subanalysis has been reported of three (3) single-center, randomized, and blinded clinical trials: (1) TAC-HFT (Transendocardial Autologous Mesenchymal Stem Cells and Mononuclear Bone Marrow Cells in Ischemic Heart Failure Trial); (2) POSEIDON (A Phase I/II, Randomized Pilot Study of the Comparative Safety and Efficacy of Transendocardial Injection of Autologous Mesenchymal Stem Cells Versus Allogeneic Mesenchymal Stem Cells in Patients With Chronic Ischemic Left Ventricular Dysfunction Secondary to Myocardial Infarction); and (3) POSEIDON-DCM (Percutaneous Stem Cell Injection Delivery Effects on Neomyogenesis in Dilated Cardiomyopathy). Baseline and 1-year cardiac structure and function and quality-of-life data were compared in a post hoc pooled analysis including ICM (n=46) and DCM (n=33) patients who received autologous or allogeneic mesenchymal stem cells. Ejection fraction improved in DCM by 7% (within-group, P=0.002) compared to ICM (1.5%; within-group, P=0.14; between-group, P=0.003). Similarly, stroke volume increased in DCM by 10.59 mL (P=0.046) versus ICM (0.2 mL; P=0.73; between-group, P=0.02). End-diastolic volume improved only in ICM (10.6 mL; P=0.04) and end-systolic volume improved only in DCM (17.8 mL; P=0.049). The sphericity index decreased only in ICM (0.04; P=0.0002). End-diastolic mass increased in ICM (23.1 g; P<0.0001) versus DCM (4.1 g; P=0.34; between-group, P=0.007). The 6-minute walk test improved in DCM (31.1 m; P=0.009) and CM (36.3 m; P=0.006) with no between-group difference (P=0.79). The New York Heart Association class improved in DCM (P=0.005) and ICM (P=0.02 between-group P=0.20). The Minnesota Living with Heart Failure Questionnaire improved in DCM (19.5; P=0.002) and ICM (6.4; P=0.03; d between-group difference 1=0.042) patients. Mesenchymal stem cell therapy was beneficial in DCM and ICM patients, despite variable effects on cardiac phenotypic outcomes. Whereas cardiac function improved preferentially in DCM patients, ICM patients experienced reverse remodeling. Mesenchymal stem cell therapy enhanced quality of life and functional capacity in both etiologies. Tompkins et al., “Comparison of Mesenchymal Stem Cell Efficacy in Ischemic Versus Nonischemic Dilated Cardiomyopathy” J Am Heart Assoc. 7:e008460 (2018).

C. Diabetes Injury Treatment

1. Ocular Injuries

The use of multipotent mesenchymal stem cells (MSCs) has been reported for the treatment of numerous degenerative disorders including the eye. In retinal degenerative diseases, MSCs exhibit the potential to regenerate into retinal neurons and retinal pigmented epithelial cells in both in vitro and in vivo studies. Delivery of MSCs was found to improve retinal morphology and function and delay retinal degeneration. In some embodiment, MSCs may reverse the pathological conditions of various ocular disorders such as age-related macular degeneration (AMD), retinitis pigmentosa, diabetic retinopathy, and glaucoma. Current stein cell treatment, however, is limited by the present understanding of MSCs mechanism of action. Ding et al., “Cellular Reparative Mechanisms of Mesenchymal Stem Cells for Retinal Diseases” Int. J. Mol. Sci. 18(1406):1-19 (2017).

Stem cells hold an multi-germ layer differentiation potential that can be directed to form almost any cell type of the body. Nicoara et al., “Novel strategies for the improvement of stem cells' transplantation in degenerative retinal diseases” Stem Cells Int. 2016:1236721 (2016). Several clinical trials have advanced to evaluate the efficiency and safety of RPE-derived human embryonic stein cells (ESCs) on patients suffering from AMD (NCT01674829) or Stargardt's Macular Dystrophy (NCT01345006). The results of the first clinical study was not reported, however, the latter indicated that the patients benefitted from the transplantation and acquired general and peripheral visions by 8-20 points. The investigators further suggested that RPE-derived hESCs could be a potential therapeutic cells that posed no evidence of unfavorable proliferation, immune-rejection, or uneventful systemic and ocular pathological conditions for a period of 22 months following to subretinal transplantation. In another recent study, preliminary data on Phase 1/II trial (NCT01344993) reported that patients affected by AMD demonstrated improvement in visual acuity after a year following allogeneic transplantation of pigmented epithelial cells derived from hESCs without any evidence of adverse effect or tumor formation related to the transplanted cells. The results demonstrated that 13 out of the 18 patients showed reconstitution in the RPE structure and improvement in the functional activity. Schwartz et al., “Human embryonic stem cell-derived retinal pigment epithelium in patients with age-related macular degeneration and Stargardt's macular dystrophy: Follow-up of two open-label phase 1/2 studies” Lancet 385:509-516 (2015).

Pre-clinical and clinical trials regarding the administration of MSCs have revealed significant restoration of the visual system through MSC-mediated therapeutic mechanisms involving (i) cell differentiation and trans-differentiation processes to replace loss or damaged cells, (ii) paracrine action for cell repair and revival, (iii) modulation of host's immune responses at inflamed site, and (iv) anti-angiogenic trophic action in certain ocular disorders. Mok et al., “Cellular mechanisms of emerging applications of mesenchymal stem cells” Malays. J. Pathol. 35:17-32 (2013); Kim et al., “Retinal angiogenesis effects of TGF-1 and paracrine factors secreted from human placental stem cells in response to a pathological environment” Cell Transplant. 25:1145-1157 (2016); and Zhao et al., “Therapeutic effects of mesenchymal stem cells administered at later phase of recurrent experimental autoimmune uveitis” Int. J. Ophthalmol. 9:1381-1389 (2016).

Dysregulation of the intraocular immune system is a pathological condition commonly manifested in AMD, glaucoma, diabetic retinopathy, and uveitis. Perez et al., “Immune mechanisms in inflammatory and degenerative eye disease” Trends Immunol 36:354-363 (2015). It is represented by a profound release of pro-inflammatory cytokines, chemokines, Matrix Metalloproteinases (MMPs) that progressively results in the loss of endothelium tight junction proteins, destruction, and leakage of BRB, hence, facilitating the infiltration of immune cells. Klaassen et al., “Molecular basis of the inner blood-retinal barrier and its breakdown in diabetic macular edema and other pathological conditions” Prog. Retin. Eye Res. 34:19-48 (2013). It is also believed that the privileged status of the eye may be compromised when predisposed to autoimmune reaction against self-antigens, for instance, uveal melanin, arrestin, interphotoreceptor retinoid-binding protein (IRBP), and recoverin, which are expressed in the retina, lens, and cornea. Forrester et al., “Good news-bad news: The yin and yang of immune privilege in the eye” Front. Immunol 3:338 (2012). This, in turn, will trigger subset of antigen-activated Cluster of Differentiation 4 (CD4) T cells to release transcription factors that are essential for downstream activation of autoimmune-associated T helper cell proliferation, such as IL-12 and Interferon- (IFN-) for T helper type 1 (Th1) cells and IL-6, IL-21, IL-23, and Transforming Growth Factor (TGF-) for Th17 cells. Caspi, R. R., “A look at autoimmunity and inflammation in the eye” J. Clin. Investig. 120:3073-3083 (2010).

2. Islet Cell Injury

Although the muscle is one of the preferable transplant sites in islet transplantation, its transplant efficacy is poor, one study reported as to whether an intramuscular co-transplantation of mesenchymal stem cells (MSCs) could improve the clinical outcome. Murine islets were co-cultured with MSCs and then analyzed the morphological changes, viability, insulin-releasing function (represented by the stimulation index), and gene expression of the islets. Five hundred (500) islets were then transplanted intramuscularly with or without 5×10⁵ MSCs to diabetic mice and measured: i) blood glucose level; ii) glucose tolerance test parameters; and iii) plasma IL-6 levels. Inflammation, apoptosis, and neovascularization in the transplantation site were evaluated histologically. The destruction of islets tended to be prevented by co-culture with MSCs. The stimulation index was significantly higher in islets co-cultured with MSCs (1.78±0.59 vs. 7.08±2.53; p=0.0025). In terms of gene expression, Sult1c², Gstm1, and Rab37 were significantly upregulated in islets co-cultured with MSCs. Although MSCs were effective in the in vitro assays, they were only partially effective in facilitating intramuscular islet transplantation. Co-transplanted MSCs prevented an early inflammatory reaction from the islets (plasma IL-6; p=0.0002, neutrophil infiltration; p=0.016 inflammatory area; p=0.021), but could not promote neovascularization in the muscle, resulting in the failure of many intramuscular transplanted islets to engraft. In conclusion, co-culturing and co-transplanting MSCs is potentially useful in islet transplantation, especially in terms of anti-inflammation, but further augmentation for an anti-apoptosis effect and neovascularization is necessary. Yoshimatsu et al., “The Co-Transplantation of Bone Marrow Derived Mesenchymal Stem Cells Reduced Inflammation in Intramuscular Islet Transplantation” PLoS ONE 10(2):e0117561 ((2015).

Islet cell transplantation is a therapy for diabetes mellitus (DM). However, the limited availability of purified islets for transplantation and the risk of immunological rejection severely limit its use. In vitro transdifferentiation of autologous bone marrow-derived mesenchymal stem cells (BMSCs) into insulin-producing cells (IPCs) could provide an abundant source of cells for this procedure and avoid immunological rejection. BMSCs were isolated and characterized and induced their in vitro differentiation into IPCs. Reverse-transcription polymerase chain reaction analysis revealed that these IPCs could express Ins1, Ins2, glucagon, glucose transporter 2, and pancreatic duodenal homeobox-1. Insulin production by the IPCs was confirmed by immunocytochemistry and Western blot analysis. On this basis, donor rats supplying BMSCs were made diabetic by a single intraperitoneal injection of streptozotocin. The IPCs were then autologously transplanted into the duodenal submucosa of diabetic rats. Grafted cells could be visualized in sections after 2, 4, and 8 weeks by immunohistochemical staining for insulin. Furthermore, in the IPC-implanted group, hyperglycemia was normalized, compared with a persistent increase in glucose levels in the diabetic group and intraperitoneal glucose tolerance test-induced responses were observed in the IPC-implanted group. These results on autologous transplantation of IPCs derived from BMSCs into the duodenal wall could offer a novel potential therapeutic protocol for DM. Zhang et al., “Insulin-Producing Cells Derived from Rat Bone Marrow and Their Autologous Transplantation in the Duodenal Wall for Treating Diabetes” Anatom. Rec. 292:728-735 (2009).

D. Neurological Degradation Injury

Due to the limited capacity of the CNS for regeneration, more effective treatment of chronic degenerative and inflammatory neurological conditions, but also of acute neuronal damage from injuries or cerebrovascular diseases, could be only achieved, theoretically at least, by stem cells that may have the potential to either regenerate or to support the survival of the existing, partially damaged, cells. A small number of stem cells are found in the adult brain in very specific regions, but this intrinsic stem cell repertoire is rather small and does not contribute significantly to the repair of damaged tissues. Transplantation of stem cells has long been suggested as a possible logical approach for repair of the damaged nervous system. Embryonic cells carrying the pluripotent and self-renewal properties represent the prototype of stem cells, but there are additional somatic stem cells that may be harvested and expanded from various tissues during adult life, such as the mesenchymal stem cells (MSC), which offer several practical advantages for the clinical application. MSC can be obtained from every adult and there are effective culture protocols for their expansion to large numbers for clinical uses. They seem to carry fewer risks for malignancies and some initial indications of their short-term safety (upon system delivery), in clinical settings, exist in the literature. Therefore, in most of the registered clinical trials with stem cells, MSC is the primary stem cell population used. This review summarizes the rationale, the mechanisms and the worldwide clinical experience with MSC, in neurological and other diseases. Kassis et al., “Mesenchymal stem cells in neurological diseases” Clin. Invest. 3(2):173-189 (2013).

Several reports have suggested the potential of various stem cell populations to induce regeneration in animal models of acute neuronal injury (such as following vascular events or acute traumatic injury), inflammatory neurological autoimmune conditions, primary CNS degenerative diseases such as Parkinson's disease (PD), Huntington's disease (HD), multiple system atrophy (MSA), amyotrophic lateral sclerosis (ALS) and Alzheimer's disease (AD) and other genetic diseases. Buhnemann et al., “Neuronal differentiation of transplanted embryonic stem cell-derived precursors in stroke lesions of adult rats” Brain 129(Pt 12), 3238-3248 (2006); Fan et al., “Endothelial progenitor cell transplantation improves long-term stroke outcome in mice” Ann. Neurol. 67(4), 488-497 (2010); Li et al., “Human marrow stromal cell therapy for stroke in rat: neurotrophins and functional recovery” Neurology 59(4):514-523 (2002); Kassis et al., “Neuroprotection and immunomodulation with mesenchymal stem cells in chronic experimental autoimmune encephalomyelitis” Arch. Neurol. 65(6):753-761 (2008); Zappia et al., “Mesenchymal stem cells ameliorate experimental autoimmune encephalomyelitis inducing T-cell anergy” Blood 106(5):1755-1761 (2005); Zhang et al., “Human bone marrow stromal cell treatment improves neurological functional recovery in EAE mice” Exp. Neurol. 195(1):16-26 (2005); Corti et al., “Wild-type bone marrow cells ameliorate the phenotype of SOD1-G93A ALS mice and contribute to CNS, heart and skeletal muscle tissues” Brain 127(Pt 11):2518-2532 (2004); Karumbayaram et al., “Human embryonic stem cell-derived motor neurons expressing SOD1 mutants exhibit typical signs of motor neuron degeneration linked to ALS” Dis. Model Mech. 2(3-4):189-195 (2009); Zhao et al., “Human mesenchymal stromal cells ameliorate the phenotype of SOD1-G93A ALS mice; Cytotherapy 9(5):414-426 (2007)’ Ben-Hur et al, “Transplantation of human embryonic stem cell-derived neural progenitors improves behavioral deficit in Parkinsonian rats” Stem Cells 22(7):1246-1255 (2004); Brederlau et al., “Transplantation of human embryonic stem cell-derived cells to a rat model of Parkinson's disease: effect of in vitro differentiation on graft survival and teratoma formation” Stem Cells 24(6), 1433-1440 (2006); Li et al., “Treatment of Parkinson disease with C17.2 neural stem cells overexpressing NURR1 with a recombined republic-deficit adenovirus containing the NURR1 gene” Synapse 61(12):971-977 (2007); Park et al., “Neuroprotective effect of human mesenchymal stem cells in an animal model of double toxin-induced multiple system atrophy parkinsonism” Cell Transplant. 20(6):827-835 (2011); Park et al., “Mesenchymal stem cells therapy exerts neuroprotection in a progressive animal model of Parkinson's disease” J. Neurochem. 10⁷(1): 141-151 (2008); and Nakao et al., “Embryonic striatal grafts restore neuronal activity of the globus pallidus in a rodent model of Huntington's disease” Neuroscience 88(2): 469-477 (1999).

### IV. Pharmaceutical Compositions

The present invention further provides pharmaceutical compositions (e.g., comprising the MSCs described above). The pharmaceutical compositions of the present invention may be administered in a number of ways depending upon whether local or systemic treatment is desired and upon the area to be treated. Administration may be topical; intratracheal, intranasal, epidermal, transdermal, oral or parenteral. Parenteral administration includes intravenous, intra-arterial, subcutaneous, intraperitoneal, intramuscular, intra-articular, intracranial, intrathecal, intrabursal, intratendonal, intralesional, perilesional, intratendon sheath, or intraventricular. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC₅₀s found to be effective in in vitro and in vivo animal models or based on the examples described herein. In general, dosage is from 1,000 to 10,000,000,000 cells per administration, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compound is administered in maintenance doses, ranging from 1,000 to 10,000,000,000 cells per administration, once or more daily, to once every 20 years.

### EXPERIMENTAL

### Example I

### Bone Marrow Collection, Processing and Expansion

An equine (e.g., a horse) was sedated with xylazine hydrochloride, the sternum was aseptically prepared, and an 11 g jamshidi needle used to collect a minimum of 360 mls of bone marrow. The needle was advanced into the 4th sternabrae approximately 2 cm, and bone marrow was then collected with negative aspiration pressure and the needle advanced 0.5 cm after 15 mls of bone marrow was collected until the needle has been advanced 3 additional centimeters.

This process was repeated moving the jamshidi caudal in the 4th sternabrae approximately 1 cm and repeating the process until 360 mls is obtained. The bone marrow was prevented from clotting by the addition of 5000 units of heparin per 30 mls of bone marrow collected.

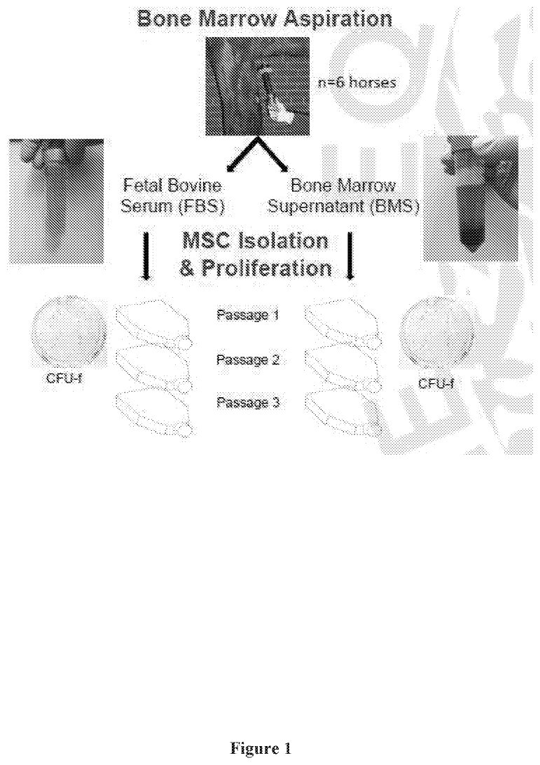

After collection, the bone marrow was processed aseptically in a fume hood to prevent contamination. The raw marrow was centrifuged at 300 g for 5 minutes, and the supernatant collected and passed through a 100 micron filter. The marrow was then combined again, and the process repeated once more. After all BMS was collected, the raw marrow undergoes red blood cell lysis and is plated to a culture flask and maintained in culture with media supplemented with 10% BMS by volume. See, FIG. 1.

### Example II

### Animals

The experimental animal of choice were female horses of the Quarter Horse breed and the age median age was 11.5 years (range 3-17). Radiographic or other diagnostic imaging of the joints was performed to verify and monitor osteoarthritic conditions.

### Example III

### Intra-Articular MSC Injection

Horses were sedated with 0.4 mg/kg xylazine hydrochloride (Lloyd Inc., Shenandoah, Iowa, USA) (100 mg/mL). The lateral aspect of each metacarpophalangeal joint was aseptically prepared. Immediately prior to joint injection, vials containing MSCs previously frozen in 95% autologous serum and 5% DMSO or MSC proliferation media alone were thawed in a 37° C. water bath. A 20-gauge, 1-inch needle was inserted into a joint distal to the lateral collateral sesamoidean ligament and 2 mL of synovial fluid was collected and transferred to tubes containing EDTA for cytological analysis. Following synovial fluid collection, MSC injection was performed as appropriate for each joint with approximately 10 million MSCs suspended in 1 mL of 95% autologous serum and 5% DMSO.

### Example IV

### Clinical Evaluations

Horses were monitored by routine physical examination and assessed for lameness at a walk every 12 hours for the week following each intra-articular injection. If any horse had lameness visible at a walk for ≥24 hours, rescue analgesia was administered (4.4 mg/kg phenylbutazone, intravenously (IV) every 24 hours) until lameness was no longer visible at a walk. Assessment of lameness was done with an objective inertial sensor system (Lameness Locator®) on the same days and immediately prior to synovial fluid collection from injected joints.

### Example V

### Cytokine Assays

The cytokines reported herein were measured by assays well known to those of skill in the art, generally using commercially available kits.

### Example VI

### Statistical Analysis

Data were imported into a commercial statistical software program (SAS, version 9.4, SAS Institute Inc., Cary, N.C., USA) for analysis. Repeated measures analysis of variance (ANOVA) was used to compare temporal changes in the biochemical and clinical outcome variables for each pair of treatment groups, with horse considered a random effect. These analyses were performed using PROC MIXED, and an autoregressive correlation structure was specified. Treatment group, time point, and their interaction were included as factors in the ANOVA. The Wilcoxon rank-sum test was used to compare population doubling time between the autologous serum-treated MSCs and the BMS-treated MSCs. For all analyses, p values<0.05 were considered significant.

We claim:

1. A method, comprising:
a) collecting a bone marrow sample from a subject;
b) centrifuging said bone marrow sample to separate a first layer comprising mesenchymal stem cells and a second layer comprising a bone marrow supernatant;
c) adding said bone marrow supernatant to a cell culture media, wherein a supplemented cell culture media is created that is xenogen-free;
d) proliferating said mesenchymal stem cells in said supplemented, xenogen-free cell culture media to create an expanded xenogen-free mesenchymal stem cell population.

2. The method of claim 1, wherein said expanded xenogen-free mesenchymal stem cell population does not contain a fetal bovine serum immunogen.

3. The method of claim 1, wherein said method further comprises incorporating said expanded xenogen-free mesenchymal stem cell population into a pharmaceutically acceptable composition.

4. The method of claim 1, wherein said subject is a mammal selected from the group consisting of human, equine, caprine, bovine, and ovine.

5. A method, comprising:
a) providing;
i) a subject comprising an osteoarthritis injury; and
ii) a pharmaceutically acceptable composition comprising an expanded xenogen-free mesenchymal stem cell population;
b) administering said pharmaceutically acceptable composition to said subject.

6. The method of claim 5, wherein said osteoarthritis injury completely heals within one year after said administering.

7. The method of claim 5, wherein said administering comprises an injection selected from the group consisting of an intra-articular injection, an intramuscular injection, an extracellular tendon injection, an intravenous injection, an intrathecal injection, an intrabursal injection, an intra-tendon sheath injection, an intraperitoneal injection, an intralesional injection, a perilesional injection, and a subcutaneous injection.

8. The method of claim 6, wherein said completely healed subject returns to pre-injury work or performance.

9. The method of claim 5, wherein said subject is a mammal selected from the group consisting of human, equine, caprine, bovine, and ovine.

10. The method of claim 5, wherein said expanded xenogen-free mesenchymal stem cell population does not contain a fetal bovine serum immunogen.

11. The method of claim 5, wherein said expanded xenogen-free mesenchymal stein cell population does contain a bone marrow supernatant.

12. The method of claim 5, wherein said osteoarthritis injury comprises a hip joint injury, a knee joint injury, an ankle joint injury, an elbow joint injury, a finger joint injury, and a toe joint injury.

13. A method, comprising:
a) providing;
i) a subject comprising a cardiovascular injury; and
ii) a pharmaceutically acceptable composition comprising an expanded xenogen-free mesenchymal stem cell population;
b) administering said pharmaceutically acceptable composition to said subject.

14. The method of claim 13, wherein said cardiovascular injury completely heals within one year after said administering.

15. The method of claim 13, wherein said administering comprises an injection selected from the group consisting of an intra-articular injection, an intramuscular injection, an extracellular tendon injection, an intravenous injection, an intrathecal injection, an intrabursal injection, an intra-tendon sheath injection, an intraperitoneal injection, an intralesional injection, a perilesional injection, and a subcutaneous injection.

16. The method of claim 14, wherein said completely healed subject returns to pre-injury work or performance.

17. The method of claim 13, wherein said subject is a mammal selected from the group consisting of human, equine, bovine, caprine, and ovine.

18. The method of claim 13, wherein said expanded xenogen-free mesenchymal stem cell population does not contain a fetal bovine serum immunogen.

19. The method of claim 13, wherein said expanded xenogen-free mesenchymal stem cell population does contain a bone marrow supernatant.

20. The method of claim 13, wherein said cardiovascular injury is selected from the group consisting of a cardiomyopathy injury, a vascular abrasion injury, a myocardial infarction injury, and a cardiac muscle injury

21. A method, comprising:
a) providing;
i) a subject comprising a diabetes injury; and
ii) a pharmaceutically acceptable composition comprising an expanded xenogen-free mesenchymal stem cell population;
b) administering said pharmaceutically acceptable composition to said subject.

22. The method of claim 21, wherein said diabetes injury completely heals within one year after said administering.

23. The method of claim 21, wherein said administering comprises an injection selected from the group consisting of an intra-articular injection, an intramuscular injection, an extracellular tendon injection, an intravenous injection, an intrathecal injection, an intrabursal injection, an intra-tendon sheath injection, an intraperitoneal injection, an intralesional injection, a perilesional injection, and a subcutaneous injection.

24. The method of claim 22, wherein said completely healed subject returns to pre-injury work or performance.

25. The method of claim 21, wherein said subject is a mammal selected from the group consisting of human, equine, bovine, caprine, and ovine.

26. The method of claim 21, wherein said expanded xenogen-free mesenchymal stem cell population does not contain a fetal bovine serum immunogen.

27. The method of claim 21, wherein said expanded xenogen-free mesenchymal stem cell population does contain a bone marrow supernatant.

28. The method of claim 21, wherein said diabetes injury is selected from the group consisting of a pancreatic injury, a pancreatic beta-cell injury, an ocular injury, and an epithelial cell injury.

29. A method, comprising:
a) providing;
i) a subject comprising a neurodegenerative injury; and
ii) a pharmaceutically acceptable composition comprising an expanded xenogen-free mesenchymal stem cell population;
b) administering said pharmaceutically acceptable composition to said subject.

30. The method of claim 29, wherein said neurodegenerative injury completely heals within one year after said administering.

31. The method of claim 29, wherein said administering comprises an injection including, but not limited to, an intra-articular injection, an intramuscular injection, an extracellular tendon injection, an intravenous injection, an intrathecal injection, an intrabursal injection, an intra-tendon sheath injection, an intraperitoneal injection, an intralesional injection, a perilesional injection, and a subcutaneous injection.

32. The method of claim 31, wherein said completely healed subject returns to pre-injury work or performance.

33. The method of claim 29, wherein said subject is a mammal that is selected from the group consisting of human, equine, bovine, caprine, and ovine.

34. The method of claim 29, where said expanded xenogen-free mesenchymal stem cell population does not contain a fetal bovine serum immunogen.

35. The method of claim 29, wherein said expanded xenogen-free mesenchymal stem cell population does contain a bone marrow supernatant.

36. The method of claim 29, wherein said neurodegenerative injury is selected from the group consisting of an inflammatory neurological autoimmune condition, a primary CNS degenerative disease, Parkinson's disease (PD), Huntington's disease (HD), multiple system atrophy (MSA), amyotrophic lateral sclerosis (ALS), and Alzheimer's disease (AD).

37. A xenogen-free cell culture medium, comprising
a) a xenogen-free, physiologically-balanced mixture of salts;
b) a xenogen-free, mixture of essential amino acids; and
c) a xenogen-free supplement.

38. The cell culture medium of claim 37, wherein said salt mixture and said essential amino acid mixture is a minimum essential media.

39. The cell culture media of claim 37, wherein said xenogen-free supplement is a bone marrow supernatant.

40. The cell culture media of claim 37, wherein said xenogen-free supplement is not a fetal bovine serum.
